(19) United States
(12) Patent Application Publication
(10) Pub. No.: US 2024/0215799 A1
(43) Pub. Date: Jul. 4, 2024

(54) IN VIVO DEVICE AND A COMBINED IMAGER THEREFOR

(71) Applicant: Given Imaging LTD., Yoqneam (IL)

(72) Inventors: Yaniv Birnboim, Shaarey Tiqwa (IL); Arkadiy Morgenshtein, Haifa (IL); Avishai Adler, Haifa (IL)

(21) Appl. No.: 18/558,266

(22) PCT Filed: May 4, 2022
(86) PCT No.: PCT/IL2022/050467

§ 371 Date: Oct. 31, 2023

Related U.S. Application Data
(60) Provisional application No. 63/186,259, filed on May 10, 2021.

(51) Int. Cl. A61B 1/00; A61B 1/04; A61B 1/06

(52) U.S. Cl. CPC A61B 1/00009; A61B 1/041; A61B 1/0638

(57) ABSTRACT

An in-vivo devices (600) includes a combined sensor array (600) having a first sensor array sensitive to a first wavelength range and a second sensor array sensitive to a second wavelength range, where the second wavelength has a partial overlap with the first wavelength range, the first sensor array is configured for collecting light in the first wavelength range and outputting a corresponding first signal, and the second sensor array is configured for collecting light in the second wavelength range and outputting a corresponding second signal. The in-vivo device further includes a processor (630) configured for receiving the first signal and the second signal, manipulating the first signal based on at least a part of the second signal corresponding to the partial overlap to output a first image, and outputting a second image based on the second signal.

### CROSS-REFERENCE TO RELATED APPLICATIONS

The present applications claims the benefit of and priority to U.S. Provisional Application No. 63/186,259, filed May 10, 2021, which is hereby incorporated by reference herein in its entirety.

### TECHNOLOGICAL FIELD

The present disclosure relates to image sensors and, more particularly, to image sensors operating with different bandwidth ranges.

### BACKGROUND

Imaging of an object involves a collection of points from the plane of the object focused by an optical system onto a collection of points on the plane of an image sensor. When there is a need to obtain spectral information as well as spatial information of the object, there is a fundamental problem, since this task involves simultaneously capturing a two-dimensional image of the object plane together with the color of each point of the object plane, which is essentially a “third dimension” of the object plane, and then recording these three dimensions of information on the two-dimensional plane of the image sensor. A number of proposed solutions have been suggested in the prior art to solve this problem.

One of the possible optical systems may be one that includes an array of pinholes that may be positioned at the focal plane of the light reflected off the object, while the image sensor is located beyond the focal point such that the image acquired is defocused (and would be later focused by appropriate software). The pinhole array is used to differentiate between points from the plane of the object, such that there would not be any overlap of points in the plane of the image sensor. Without the pinholes, there is overlap between points on the imager's plane, which would make it practically impossible to correlate between points on the imager's plane to points on the object's plane and, thus, practically impossible to restore the spatial information of the object.

A filter array comprising sub-filters may be added to the system and may be positioned at the aperture stop, such that spectral information may be acquired by the optical system as well as spatial information. That is, every pixel at the imager's plane has two “coordinates”; one for the angle at which light was reflected off the object, and a second for the sub-filter which the light reflecting off the object passed through. However, the main disadvantages of using a pinhole array are losing spatial information and losing light when collecting the light reflected off the object, since the pinhole array blocks some of the light reflected off the object from being projected onto the imager.

Another possible optical system that may be used to create an image of an object while providing spatial and spectral information is one where, instead of a filter array located at the aperture stop, a mask is located at the aperture stop. Such an optical system does not include a pinhole array, so there is an overlap between pixels of the image sensor. The mask is random with the requirement of being 50% open for passage of light that is reflected off the imaged object. With this optical system, there is minimal loss of spatial resolution, since the scenes that are being imaged do not consist of dramatic spectral changes, and the objects are relatively large so it is not difficult to distinguish between areas of the same spectra.

The mask, according to the above optical system, provides combinations of spatial and spectral “coordinates” that may describe the object. (The “coordinates” are acquired by the imager followed by software reconstruction, in order to focus the acquired images). In areas of the object where the spectrum is substantially similar, only the spatial data is missing. The mask is then used to separate between close points with similar spectrum on the imager's plane, so it would be easier to correlate those points to points on the object's plane. However, when close points on the object have different spectrum (e.g., along the edges of the object) it is more difficult to distinguish between the points projected onto the imager.

Images that provide spatial as well as spectral information may be important within small scale in-vivo imaging devices, e.g., endoscopes and capsule endoscopes. Spatial information is needed in order to determine the in-vivo location of the device, and spectral information of in-vivo tissue is important for determining various diseases at early stages that may be expressed in changes in spectra of various in-vivo particles, e.g., hemoglobin. There is, therefore, interest in a new optical system that may be implemented into devices that are to be inserted in-vivo, in order to acquire images that contain both spatial and spectral information.

The descriptions in the above paragraphs herein are not to be inferred as meaning that they are in any way relevant to the patentability of the presently disclosed subject matter.

### SUMMARY

As used herein, the term “light” may refer to electromagnetic radiation in the visible spectrum and/or electromagnetic radiation in the infrared spectrum, depending on the context.

In accordance with aspects of the present disclosure, an in-vivo device includes: a combined sensor array having a first sensor array sensitive to a first wavelength range and a second sensor array sensitive to a second wavelength range, where the second wavelength range has a partial overlap with the first wavelength range, the first sensor array is configured for collecting light in the first wavelength range and outputting a corresponding first signal, and the second sensor array is configured for collecting light in the second wavelength range and outputting a corresponding second signal. The in-vivo device further includes a processor configured for: receiving the first signal and the second signal, manipulating the first signal based on at least a part of the second signal corresponding to the partial overlap to output a first image, and outputting a second image based on the second signal.

The term “partial overlap” should be understood with reference to the first wavelength range, i.e., how much of the first wavelength range is overlapped by the second wavelength range. In accordance with some examples, the following variations are applicable: the second wavelength range is completely contained within the first wavelength range and overlaps the beginning or the end of the first wavelength range; the second wavelength range is completely contained within the first wavelength range and overlaps a middle portion of the first wavelength range; and the second wavelength range has a portion not overlapping with the first wavelength range.

In all three cases, a portion of the first wavelength range does not overlap with the second wavelength range.

In various embodiments of the in-vivo device, the first sensor array includes RGB sensors, and the second sensor array includes infrared sensors. In accordance with one example, the second wavelength range includes near infrared.

In various embodiments of the in-vivo device, the manipulation of the first signal based on at least a part of the second signal may be in the form of Boolean operations between the first and the second signal. In various embodiments of the in-vivo device, the manipulation of the first signal based on at least a part of the second signal may involve subtraction, addition, superposition, phase change, etc. In accordance with a particular example, the overlapping portion of the second signal may be subtracted from the first signal to leave a modified first signal.

In various embodiments of the in-vivo device, the first wavelength range includes the infrared (IR) range such that the first sensor array has some sensitivity in the IR range. Thus, in accordance with a specific example, digitally subtracting the second signal from the first signal provides a cutoff effect, resulting in an RGB image having reduced light redundancy.

In various embodiments of the in-vivo device, the second signal acquired by the second sensor array is used both for outputting a second image which is an IR image, and also for digitally providing the cutoff to output the first image.

In various embodiments of the in-vivo device, the in-vivo device is a swallowable capsule endoscope.

In various embodiments of the in-vivo device, the processor is further configured to: access data indicative of at least one of: motion of the in-vivo device or turbidity around the in-vivo device, and based on the data, configure at least one of: imaging modality of the combined sensor array or frame rate of the combined sensor array.

In accordance aspects of the present disclosure, a method is disclosed for obtaining images by an in-vivo device having a processor and a combined sensor array that includes a first sensor array sensitive to a first wavelength range and a second sensor array sensitive to a second wavelength range, where the second wavelength has a partial overlap with the first wavelength range. The method includes: using the combined sensor array to collect light in the first wavelength range and output a corresponding first signal and to collect light in the second wavelength range and output a corresponding second signal; receiving, by the processor, the first signal and the second signal; manipulating, by the processor, the first signal based on at least a part of the second signal corresponding to the partial overlap, to output a first image; and outputting, by the processor, a second image based on the second signal.

The method may also include providing, by the processor, a combined image of the first and second images. The combined image may be any one of the following: an overlay of the first and second images; a toggled image between the first and second image; and a flickering image.

In various embodiments of the method, the partial overlap corresponds to at least one of: the second wavelength range is completely contained within the first wavelength range and overlaps a beginning or an end of the first wavelength range; the second wavelength range is completely contained within the first wavelength range and overlaps a middle portion of the first wavelength range; or the second wavelength range has a portion not overlapping with the first wavelength range.

In various embodiments of the method, a portion of the first wavelength range does not overlap with the second wavelength range.

In various embodiments of the method, the first sensor array includes RGB sensors, and the second sensor array includes infrared sensors.

In various embodiments of the method, the second wavelength range includes near infrared. In various embodiments of the method, the first wavelength range includes infrared range such that the first sensor array has at least some sensitivity in the infrared range.

In various embodiments of the method, the method includes: accessing data indicative of at least one of: motion of the in-vivo device or turbidity around the in-vivo device; and based on the data, configure at least one of: imaging modality of the combined sensor array or frame rate of the combined sensor array.

The in-vivo device may be provided with illumination components configured for providing light to the GI tract configured for being reflected from the GI tract to the imager.

In accordance with a specific example, the in-vivo device may include a first illumination arrangement configured for providing illumination in a wavelength range corresponding to the wavelength range of the first sensor array, and a second illumination arrangement configured for providing illumination in a wavelength range corresponding to the wavelength range of the second sensor array.

The in-vivo device may include a controller configured for operating the first and second illumination arrangements. The controller may also be coupled to the processor and be configured for operating the illumination arrangements under different operational modes based on data received from the processor. For example, upon identifying a certain pathology in the GI tract, the processor may indicate to the controller to operate the illumination arrangements in a manner favoring one illumination arrangement over the other. The controller may also control additional illumination parameters such as light intensity and may support different illumination modalities based on the type of illumination. In addition, the controller may also control the duration of illumination and other parameters.

In accordance with a specific example, the first illumination arrangement constitutes the primary illumination arrangement, and the controller may be configured to switch to the second illumination arrangement on demand, or vice versa.

It is appreciated that the above described imager may also provide the opportunity to simultaneously acquire two images, each in a different wavelength range, without the need of physical filters.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a schematic view of an exemplary layout of an optical sensor having both RGB and IR sensor sensing elements, in accordance with aspects of the present disclosure;

FIG. 2 is an exemplary schematic spectral diagram of the RGB portion of the optical sensor shown in FIG. 1, in accordance with aspects of the present disclosure;

FIG. 3 is a schematic view of an exemplary manipulation of the spectral diagrams, in accordance with aspects of the present disclosure;

FIG. 4 shows schematic views taken of a scene through bile using IR illumination and IR imaging, at various distances, in accordance with aspects of the present disclosure;

FIG. 5 shows schematic views taken of a scene through bile using white light illumination and RGB imaging, at various distances, in accordance with aspects of the present disclosure;

FIG. 6 is a diagram of exemplary components of a capsule endoscope, in accordance with aspects of the present disclosure;

FIG. 7 is a flow diagram of an exemplary operation for outputting images, in accordance with aspects of the present disclosure;

FIG. 8 is a diagram of exemplary images captured using RGB imaging and IR imaging, in accordance with aspects of the present disclosure;

FIG. 9 is another diagram of exemplary images captured using RGB imaging and IR imaging, in accordance with aspects of the present disclosure; and

FIG. 10 is yet another diagram of exemplary images captured using RGB imaging and IR imaging, in accordance with aspects of the present disclosure.

It will be appreciated that, for simplicity and clarity of illustration, elements shown in the figures have not necessarily been drawn accurately or to scale. For example, the dimensions of some of the elements may be exaggerated relative to other elements for clarity, or several physical components may be included in one functional block or element. Further, where considered appropriate, reference numerals may be repeated among the figures to indicate corresponding or analogous elements.

### DETAILED DESCRIPTION

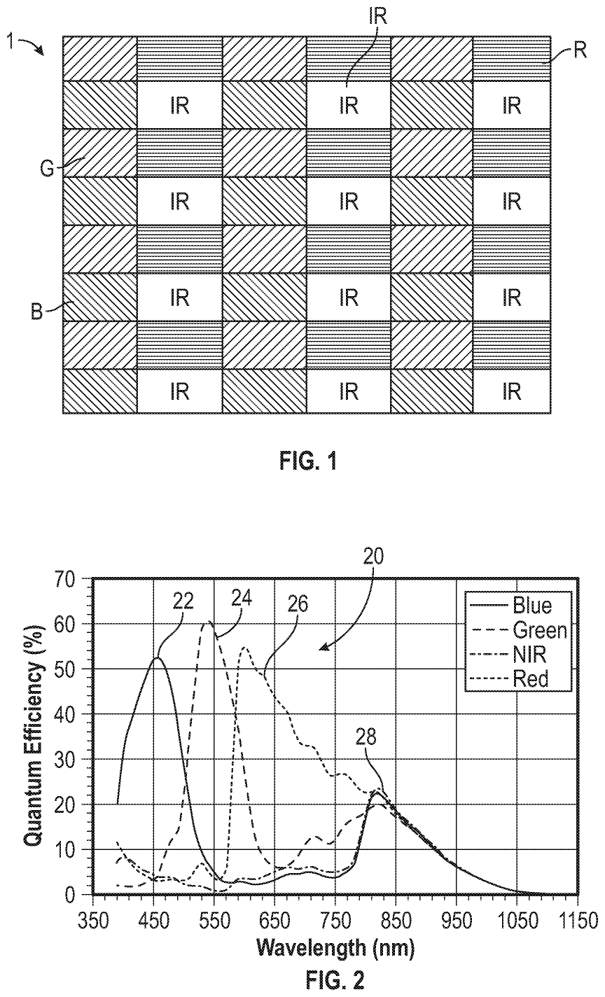

Attention is first drawn to FIG. 1, in which an RGB+IR sensor 1 is shown, including an array of sensing elements grouped into groups of four sensing elements, such that each group includes a Red sensing element R, a Green sensing element G, a Blue sensing element B, and an IR sensing element IR. This differs from the common RGB array which includes two Green sensing elements per group.

Turning now to FIG. 2, a spectral diagram 20 of the RGB portion of the sensor is shown, based only on the R, G, B sensing elements. As can be seen, the RGB portion of the sensor has an increased sensitivity in the range of 400 nm to 650 nm, after which the sensitivity declines. Specifically, the blue sensing elements have a sensitivity peak 22 around 450 nm, the green sensing elements have a sensitivity peak 24 around 540 nm, and the red sensing elements have a sensitivity peak 26 around 625 nm.

In addition, the RGB sensor also has a certain sensitivity peak 28 in the IR range, between 750 nm and 860 nm (also referred to herein as “second peak”). The IR portion of the sensor has a sensitivity around 750 nm and 860 nm, generally corresponding to the second peak of the RGB portion of the sensor.

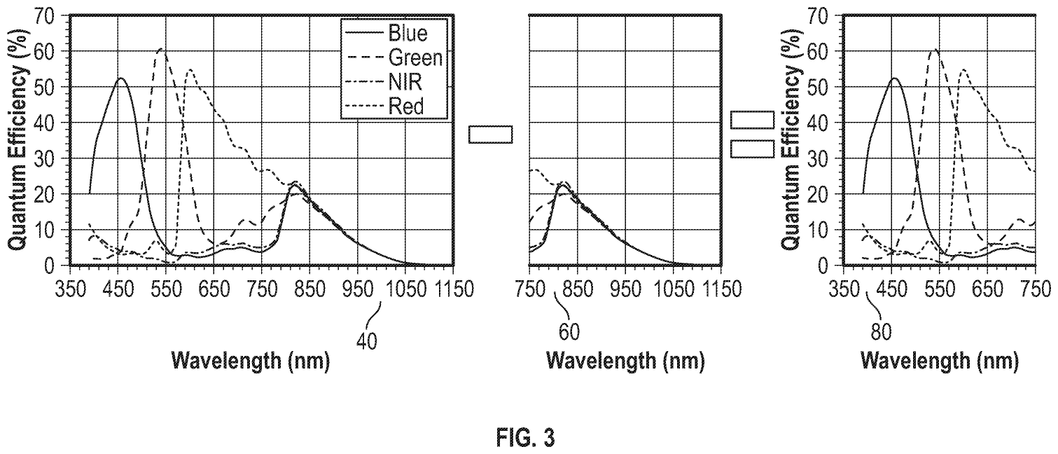

With additional reference to FIG. 3, in accordance with aspects of the present disclosure, a first image is first acquired (not shown) by the RGB portion of the sensor (not shown), using white light illumination, wherein the entire spectrum 40 of the RGB portion of the sensor is active, including the second peak 28 (FIG. 3) in the IR range. The RGB image provides a clear color image of the scene (in this case the colon section of the GI). However, the white light penetration level through the fluids of the GI is fairly low, owing to the presence of bile and other possible substances, allowing to view only a short distance ahead (for example, one or two folds ahead in case of the colon).

A second image is then acquired (not shown) by the IR sensing elements of the sensor, using IR illumination, in the narrow IR spectrum 60. While the IR image has considerably less color in it, the penetration of IR illumination is considerably higher than that of white light, allowing to see deeper (for example, two, three and even four folds ahead in case of the colon). This provides, inter alia, the advantage of overcoming GI fluids and bile and improving the visibility of images.

Once the two images have been acquired, the signals 60 of the IR image are digitally removed from the signals 40 of the first image, thereby leaving only the main RGB range, resulting in an improved RGB image, the schematic of which is shown as 80.

This provides an artificial cutoff of the RGB spectral range, removing the IR end of the RGB spectrum, this being done without requiring any physical cutoff filters. Moreover, this also provides a second, RGB image of the same site.

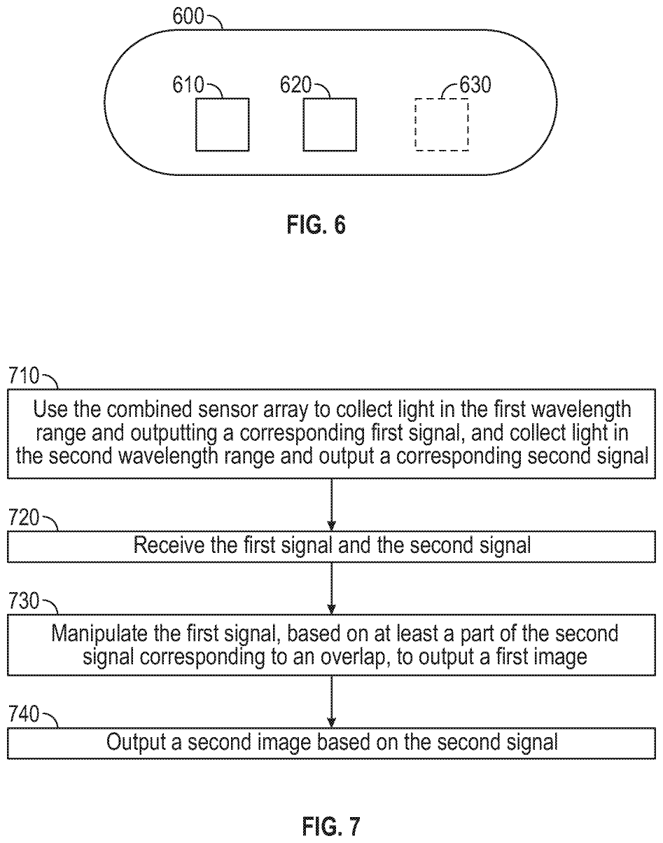

The sensor may be incorporated into an optical module used in an in-vivo device and may be configured for obtaining images in-vivo. One example of such an in-vivo device is a swallowable capsule (e.g., FIG. 6, 600) configured for obtaining images of the GI tract of a patient.

The in-vivo device may also include a processor and a controller (not shown), configured for receiving the images (both RGB and IR) received from the image sensor, perform the manipulation thereon, and indicate to the controller if any adjustments should be made to the operational modalities of the device (higher frame rate, more emphasis on a specific illumination etc.).

As a result of the above, various combinations of images may be displayed to an end user, including, but not limited to: an RGB image (40); an IR image (60); a combined RGB+IR image (40+60); a toggled view between the RGB and the IR images.

The acquisition of the IR image therefore provides two advantages, operating in complete synergy with one another: the ability to acquire a stand-alone IR image using a dedicated IR sensor; and the ability to obtain an RGB image with reduced light redundancy, without the need for a physical cutoff filter or arrangement.

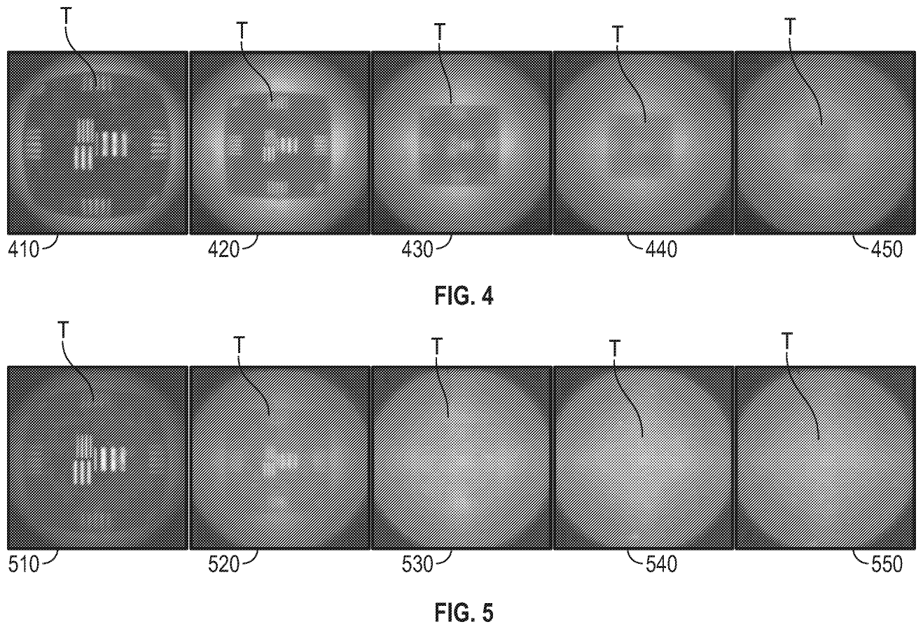

Further attention is drawn to FIG. 4 in order to illustrate the effectiveness of IR images in bile. FIG. 4 shows images taken through a bile and water medium (5 gr of bile for 100 ml of water), at 0 mm, 4 mm, 8 mm, 12 mm and 16 mm respectively. The target panel T is clearly seen in at least the 0 mm 410, 4 mm 420 and 8 mm 430 ranges, and is moderately seen at the 12 mm 440 and 16 mm 450 ranges.

In contrast, the images shown in FIG. 5 are taken through the same bile medium as FIG. 4, but using white light illumination, at the same ranges of 0 mm 510, 4 mm 520, 8 mm 530, 12 mm 540, and 16 mm 550. As can clearly be seen, the target is clearly visible at 0 mm 510, moderately visible at 4 mm 520 and barely visible in the other ranges (8 mm 530, 12 mm 540, and 16 mm 550). FIGS. 4 and 5 demonstrate the utility of using IR illumination in a bile medium.

Referring now to FIG. 6, there is shown a block diagram of components of an in-vivo device having the form of a capsule endoscope 600. The capsule endoscope 600 is configured to be swallowed by a patient and then obtain images of the GI tract of the patient. The capsule endoscope 600 may include sensors 610, a controller 620, and an optional processor 630. In various embodiments, the sensors 610 implement the combined sensor array described above herein. The controller 620 is configured to control operations of the capsule endoscope 600, including imaging operations and other operations. For clarity of illustration, not all components of the capsule endoscope are illustrated. Persons skilled in the art will recognize such other components (e.g., communication, storage, LEDs, etc.) and will understand that the controller 620 may control the operations of such other components. In various embodiments, the controller 620 and the processor 630 may be integrated into a single device, such as an application specific integrated circuit (ASIC) or a system on a chip, among other things. For convenience, the terms “controller” and “processor” may be used interchangeably, unless the context and usage indicate otherwise.

In various embodiments, the controller 620 may be configured to receive the images (both RGB and IR) from the sensors 610 and perform the operations described above, such as the operations described in connection with FIG. 3. For example, the controller 620 may control adjustments to the operational modalities of the capsule endoscope 600, such as higher frame rate, more emphasis on a specific illumination, among other things. In various embodiments, the optional processor 630 may cooperate with the sensors 610 and the controller 620 to perform certain operations described above herein, such as certain operations described in connection with FIG. 3.

The descriptions, examples, and embodiments disclosed in connection with FIG. 6 are illustration, and variations are contemplated to be within the scope of the present disclosure. For example, although FIG. 6 is described in relation to a capsule endoscope, the disclosure is applicable to other in-vivo devices as well.

FIG. 7 shows a flow chart of an operation for a combined sensor array, such as the sensors 610 of FIG. 6. The combined sensor array may include a first sensor array having sensors sensitive to a first wavelength range (e.g., RGB and IR spectrums) and a second sensor array having sensors sensitive to a second wavelength range (e.g., IR spectrum), where the second wavelength partially overlaps with the first wavelength range.

At block 710, the operation involves using the combined sensor array to collect light in the first wavelength range and outputting a corresponding first signal, and collect light in the second wavelength range and output a corresponding second signal. In various embodiments, the first signal may represent sensor readings in the RGB and IR spectrums, and the second signal may represent sensor readings in the IR spectrum.

At block 720, the operation involves receiving the first signal and the second signal by a controller and/or a processor, such as the controller 620 and/or the processor 630 of FIG. 6.

At block 730, the operation involves manipulating the first signal, based on at least a part of the second signal corresponding to the overlap, to output a first image. In various embodiments, the operation of block 730 may subtract the second signal from corresponding portions of the first signal, as described in connection with FIG. 3. The operations of block 730 may be performed by a controller and/or a processor, such as the controller 620 and/or the processor 630 of FIG. 6.

At block 740, the operation involves outputting a second image based on the second signal. In various embodiments, the second image may be the image represented by sensor readings in the IR spectrum.

The descriptions, examples, and embodiments disclosed with respect to FIG. 7 are illustrative, and variations are contemplated to be within the scope of the present disclosure.

Additional aspects of the present disclosure will be described below.

Infrared (IR) imaging provides advantages in visualization of tissue in turbid and dark situations and/or situations where a tissue feature may be confused with obstructions (e.g., dirt, debris, content, etc.) adhered to the housing of a capsule endoscope housing.

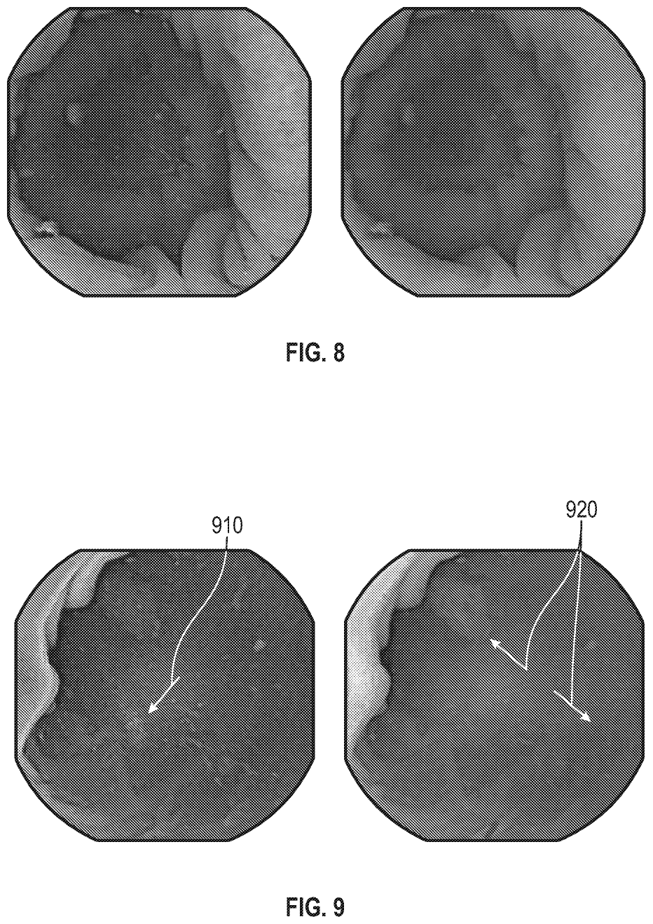

In cases of turbidity or darkness, IR allows visualization of more details through the turbidity and visualization of farther distances in the darkness (e.g., more folds in the lumen), as shown the example of FIG. 8. In FIG. 8, the left-side image was acquired using RGB imaging, and the right-side image was acquired using IR imaging. The IR image on the right side provides greater visualization of the organ walls and the lumen at farther distances.

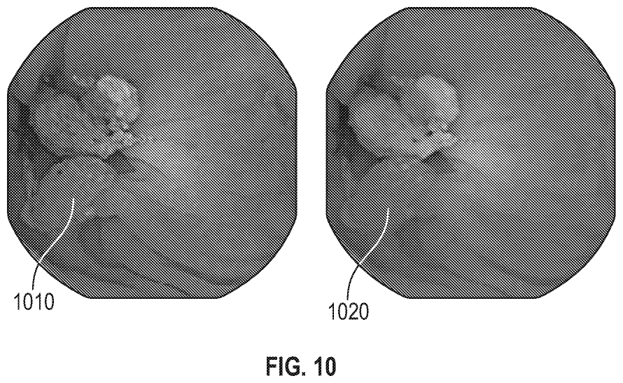

In cases where a tissue feature may be confused with obstructions (e.g., dirt, debris, content, etc.) adhered to the housing of a capsule endoscope housing, an RGB image and an IR image may be used to distinguish the tissue from the obstruction, as shown in FIGS. 9 and 10. In FIG. 9, the left-side image was acquired using RGB imaging, and the right-side image was acquired using IR imaging. The RGB image shows a feature 910 that could be a tissue feature or an obstruction adhered to the housing of the capsule. Because the IR image does not show such feature, it can be determined that the feature 910 was an obstruction adhered to the housing of the capsule. In contrast, the features 920 that appear in the IR image also appear in the RGB image, so those features 920 are tissue features rather than obstructions. As another example, in FIG. 10, the left-side image was acquired using RGB imaging, and the right-side image was acquired using IR imaging. A feature 1010 that appears in the RGB image may be a tissue feature or may be an obstruction. Because the feature 1010 also appears as a feature 1020 in the IR image, it can be determined that the feature is a tissue feature rather than an obstruction adhered to the housing of the capsule.

In various situations, the addition of IR sensor data may impact image size, as it may be data added to RGB data. The impact to image size may then impact the maximal frame rate that can be captured of a sensor array and capsule endoscope. In some situations, increased frame rate may improve the accuracy of clinical assessments based on capsule endoscopy images, such as capsule endoscopy of a colon. However, in a turbid/dark situations, increasing the frame rate may not contribute to better tissue coverage and may not improve clinical assessments. Thus, various situations may benefit from both IR imaging and higher frame rate and various situations may not.

In accordance with aspects of the present disclosure, and with reference to FIG. 6, the imaging frame rate and imaging modality of the sensors 610 may be controlled by the controller 620 based on the capsule's motion and the darkness or turbidity around the capsule. An example of such control is shown in Table 1 below.

TABLE 1

| Motion | Dark/turbid | Frame rate | Imaging modality |
|---|---|---|---|
| Lower | Lower | Lower frame rate | RGB |
| Lower | Higher | Lower frame rate/ | RGB + IR (simultaneous) |
| | | Adaptive frame rate | |
| Higher | Lower | Higher frame rate | RGB |
| Higher | Higher | Moderate frame rate/ | RGB + IR (simultaneous) |
| | | Adaptive frame rate | |

The motion of the capsule endoscope 600 and the darkness/turbidity around the capsule may be determined in various ways. Determining turbidity, as used herein, may refer to a capability to distinguish between the tissue and the other content that may obscure clear vision of the tissue.

In various embodiments, motion of the capsule endoscope 600 may be determined by a processor, such as the processor 630, by comparing the intensity of pairs of images or of elements of pairs of images, generating a variance for the compared images, and calculating the motility of the capsule from the variances, as described in U.S. Pat. No. 7,200,253, which is hereby incorporated by reference herein in its entirety. Other ways of determining motion of the capsule are contemplated to be within the scope of the present disclosure.

In various embodiments, darkness or turbidity around the capsule may be determined based on metrics such as statistical measures for a histogram of pixel brightness in an image. For example, if the mean of pixel brightness in an image is below a threshold and the variance is below a particular threshold, these metrics may reflect a turbid or dark environment around the capsule. Other ways of determining turbidity or darkness are contemplated, such as the techniques described in U.S. Pat. No. 8,861,783, which is hereby incorporated by reference herein in its entirety. In various embodiments, the motion and/or turbidity or darkness determinations may operate based on a portion of an image. In various embodiments, the motion and/or turbidity or darkness determinations may not process every image frame and may, instead, execute at a regular time interval, such as every one second, or every three seconds, or at another time interval.

As described above, the processor 630 in the capsule endoscope 600 may determine motion and/or turbidity and darkness. In various embodiments, the capsule endoscope 600 may communicate images (and optionally additional data) for a separate device or system to determine motion and/or turbidity and darkness in real time. As used herein, the term “real time” refers to processing that occurs while the capsule endoscope is still operating within the GI tract of a person. In various embodiments, the separate device or system that determines motion and/or turbidity and darkness in real time may be a wearable device that receives the images and data from the capsule endoscope 600. In various embodiments, the separate device or system that determines motion and/or turbidity and darkness in real time may be a smartphone or may be a cloud computing system that communicates with the wearable device. Other variations are contemplated to be within the scope of the present disclosure.

In accordance with aspects of the present disclosure, and with reference to Table 1, if the processor 630 or the separate device determines there is no motion above a particular threshold and there is no darkness or turbidity above a particular threshold, the frame rate of the sensors 610 can be set to a lower frame rate (e.g., 10 fps or lower) and the imaging modality can be set to RGB imaging only. If the processor 630 or the separate device determines there is no motion above a particular threshold but there is darkness or turbidity above a particular threshold, the frame rate of the sensors 610 can be set to a lower frame rate (e.g., 10 fps or lower) or an adaptive frame rate, and the imaging modality can be set to simultaneous RGB and IR imaging. The adaptive frame rate may vary the frame rate depending on the degree of turbidity or darkness. If the processor 630 or the separate device determines there is motion above a particular threshold but there is no darkness or turbidity above a particular threshold, the frame rate of the sensors 610 can be set to a higher frame rate (e.g., 40 fps or higher) and the imaging modality can be set to RGB imaging only. If the processor 630 or the separate device determines there is motion above a particular threshold and there is darkness or turbidity above a particular threshold, the frame rate of the sensors 610 can be set to a moderate frame rate between the lower frame rate and the higher frame rate (e.g., between 10 fps and 40 fps) or an adaptive frame rate, and the imaging modality can be set to simultaneous RGB and IR imaging. The adaptive frame rate may vary the frame rate depending on the degree of turbidity or darkness. The numerical examples are merely illustrative and other numerical values are contemplated. Additionally, various thresholds described above may have same value or may have different values.

Table 1 is illustrative, and variations are contemplated to be within the scope of the present disclosure. For example, in various embodiments, if the processor 630 or the separate device or system determines there is no motion above a particular threshold, then no turbidity/darkness determination is needed and the sensors 610 can be set to lower frame rate and to simultaneous RGB and IR imaging. In various embodiments, if the processor 630 or the separate device or system determines there is turbidity above a particular threshold, the imaging modality may be set to IR imaging only, without RGB imaging, which may reduce overall power consumption in the capsule endoscope 600.

In various embodiments, other factors may contribute to the setting a frame rate and imaging modality. For example, the capsule endoscope 600 and/or a separate device may determine the GI segment the capsule is located in (e.g., small bowel, colon, etc.) or may determine the amount of power remaining in the capsule 600. The frame rate and imaging modality may be determined based on such factors and/or other factors, as well.

In accordance with aspects of the present disclosure, the controller 620 may control additional components and/or features. In various embodiments, the capsule endoscope 600 may include one or more LEDs for illumination, and the controller 620 may control the LED exposure times according to operation modes. For example, the controller 620 can provide for higher current and shorter illumination in RGB-only imaging mode, when white light LEDs are used and IR LEDs are not used. When both white light and IR LEDs are active, current is shared between the LEDs and the controller 620 may provide for longer illumination period.

In various embodiments, the controller 620 may control degree of image compression based on the imaging modality. For example, a particular image compression may be used for RGB-only imaging, while a different image compression may be used for RGB with IR imaging.

In various embodiments, controller 620 may control how much data from the sensors 610 are read out. Referring also to FIG. 1, in RGB-only imaging, only the RGB triplet may be read out while the 4th IR pixel may not be read out. Doing so may decrease the readout time for the sensors 610 and may allow for beneficial increases in frame rate.

Accordingly, various features and operations of a capsule endoscope are disclosed herein. It is intended that such features and operations be applicable to in-vivo devices other than capsule endoscopes, as well.

Those skilled in the art to which the present disclosure pertains will readily appreciate that numerous changes, variations, and modifications can be made without departing from the scope of the present disclosure, mutatis mutandis.

The embodiments disclosed herein are examples of the disclosure and may be embodied in various forms. For instance, although certain embodiments herein are described as separate embodiments, each of the embodiments herein may be combined with one or more of the other embodiments herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an embodiment,” “in embodiments,” “in various embodiments,” “in some embodiments,” or “in other embodiments” may each refer to one or more of the same or different embodiments in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

Any of the herein described operations, methods, programs, algorithms, or codes may be converted to, or expressed in, a programming language or computer program embodied on a computer, processor, or machine-readable medium. The terms “programming language” and “computer program,” as used herein, each include any language used to specify instructions to a computer or processor, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, Python, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

It should be understood that the foregoing description is only illustrative of the present disclosure. To the extent consistent, any or all of the aspects detailed herein may be used in conjunction with any or all of the other aspects detailed herein. Various alternatives and modifications can be devised by those skilled in the art without departing from the disclosure. Accordingly, the present disclosure is intended to embrace all such alternatives, modifications, and variances. The embodiments described with reference to the attached drawing figures are presented only to demonstrate certain examples of the disclosure. Other elements, steps, methods, and techniques that are insubstantially different from those described above and/or in the appended claims are also intended to be within the scope of the disclosure.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

1. An in-vivo device comprising:
a combined sensor array comprising a first sensor array sensitive to a first wavelength range and a second sensor array sensitive to a second wavelength range, the second wavelength range having a partial overlap with the first wavelength range, the first sensor array configured for collecting light in the first wavelength range and outputting a corresponding first signal, and the second sensor array configured for collecting light in the second wavelength range and outputting a corresponding second signal; and
a processor configured for:
receiving the first signal and the second signal;
manipulating the first signal based on at least a part of the second signal corresponding to the partial overlap, to output a first image; and
outputting a second image based on the second signal.

2. An in-vivo device according to claim 1, wherein the partial overlap corresponds to at least one of:
the second wavelength range is completely contained within the first wavelength range and overlaps a beginning or an end of the first wavelength range;
the second wavelength range is completely contained within the first wavelength range and overlaps a middle portion of the first wavelength range; or
the second wavelength range has a portion not overlapping with the first wavelength range.

3. An in-vivo device according to claim 2, wherein a portion of the first wavelength range does not overlap with the second wavelength range.

4. An in-vivo device according to claim 1, wherein the first sensor array comprises RGB sensors and the second sensor array comprises infrared sensors.

5. An in-vivo device according to claim 4, wherein the second wavelength range includes near infrared.

6. An in-vivo device according to claim 1, wherein the first wavelength range includes infrared range such that the first sensor array has at least some sensitivity in the infrared range.

7. An in-vivo device according to claim 1, wherein the in-vivo device is a swallowable capsule endoscope.

8. An in-vivo device according to claim 1, wherein the processor is further configured to:
access data indicative of at least one of: motion of the in-vivo device or turbidity around the in-vivo device; and
based on the data, configure at least one of: imaging modality of the combined sensor array or frame rate of the combined sensor array.

9. A method for obtaining images by an in-vivo device having a processor and a combined sensor array comprising a first sensor array sensitive to a first wavelength range and a second sensor array sensitive to a second wavelength range, the second wavelength having a partial overlap with the first wavelength range, the method comprising:
using the combined sensor array to collect light in the first wavelength range and output a corresponding first signal and to collect light in the second wavelength range and output a corresponding second signal;
receiving, by the processor, the first signal and the second signal;
manipulating, by the processor, the first signal based on at least a part of the second signal corresponding to the partial overlap, to output a first image; and
outputting, by the processor, a second image based on the second signal.

10. A method according to claim 9, wherein the partial overlap corresponds to at least one of:
the second wavelength range is completely contained within the first wavelength range and overlaps a beginning or an end of the first wavelength range;
the second wavelength range is completely contained within the first wavelength range and overlaps a middle portion of the first wavelength range; or
the second wavelength range has a portion not overlapping with the first wavelength range.

11. A method according to claim 10, wherein a portion of the first wavelength range does not overlap with the second wavelength range.

12. A method according to claim 9, wherein the first sensor array comprises RGB sensors and the second sensor array comprises infrared sensors.

13. A method according to claim 12, wherein the second wavelength range includes near infrared.

14. A method according to claim 9, wherein the first wavelength range includes infrared range such that the first sensor array has at least some sensitivity in the infrared range.

15. A method according to claim 9, further comprising:
accessing data indicative of at least one of: motion of the in-vivo device or turbidity around the in-vivo device; and
based on the data, configure at least one of: imaging modality of the combined sensor array or frame rate of the combined sensor array.
